(19) United States
(12) Patent Application Publication
(10) Pub. No.: US 2025/0195909 A1
(43) Pub. Date: Jun. 19, 2025

(54) LIGHT THERAPY DEVICE AND METHOD OF PROVIDING LIGHT THERAPY

(71) Applicant: Mark Sawyer, Lake Mary, FL (US)

(72) Inventor: Mark Sawyer, Lake Mary, FL (US)

(21) Appl. No.: 18/672,404

(22) Filed: May 23, 2024

Related U.S. Application Data
(63) Continuation of application No. 18/643,452, filed on Apr. 23, 2024.
(60) Provisional application No. 63/612,237, filed on Dec. 19, 2023.
(60) Provisional application No. 63/612,253, filed on Dec. 19, 2023.

(51) Int. Cl. A61N 5/06

(52) U.S. Cl. CPC A61N 5/0613; A61N 5/0616; A61N 5/0617; A61N 5/0618; A61N 2005/0626; A61N 2005/0652; A61N 2005/0659; A61N 2005/0663

(57) ABSTRACT

A method of providing light therapy includes providing a light therapy device having a main body with an array of optical emitters, a display panel and a system controller. The array of optical emitters includes a plurality of sub arrays each having a plurality of individual optical emitters that are either a single-color LED, a multi-color LED, or near infrared LED, and each can operate at one of a fixed or user-defined wavelength. The display panel includes a touchscreen device that sends and receives information with a device user and provides a presentation screen showing a virtual representation of the output of the device. A remote control includes a display screen for remotely viewing and controlling the operation of the device. Light therapy from the device is provided across a plurality of target wavelengths via one or more default or user-defined sub arrays.

### CROSS-REFERENCE TO RELATED APPLICATIONS

This application claims the benefit of U.S. Application Ser. Nos. 63/612,237 and 63/612,253 both filed on Dec. 19, 2023, the contents of each of which are incorporated herein by reference.

### TECHNICAL FIELD

The present invention relates generally to therapy devices, and more particularly to a method of device having a plurality of optical emitters for providing light therapy to a user.

### BACKGROUND

The statements in this section merely provide background information related to the present disclosure and may not constitute prior art.

Recent scientific research has shown that exposing human skin to certain types of light can have a meaningful effect on a person's overall health. Depending on the intensity and wavelength of the light, these effects can include reducing stress, eliminating headaches, promoting hair growth, increasing collagen production, reducing wrinkles, reducing the appearance of scars, and reducing or eliminating skin acne.

For these reasons, there are several known types of light therapy devices currently on the market. Such devices typically comprise a panel having an array of non-adjustable lights that operate in an ON and OFF operating state.

Although useful, these devices typically provide a single type of light at a set wavelength. As such, because different therapeutic results are achieved at different wavelengths, these devices are unable to perform different types of light therapy at different wavelengths.

Accordingly, it would be beneficial to provide a light therapy device having an optical array that can provide light emissions at a plurality of different fixed or user-selectable wavelengths so as to conduct a plurality of different therapeutic sessions individually or simultaneously during a single session.

### SUMMARY OF THE INVENTION

The present invention is directed to providing light therapy via a light therapy device that includes a main body having an array of optical emitters, a display panel, and a system controller. In one embodiment, the array of optical emitters can include a plurality of sub arrays each having a plurality of individual optical emitters that operate at a target wavelength. Each of the optical emitters can be single color LED's multi-color LED's or near infrared LED's and can operate at one of a fixed or user-defined wavelength.

In one embodiment, the display panel includes a touchscreen device that can send and receive information with a device user. The touchscreen device can allow a user to control the operation of the array and can allow the user to select individual sub arrays and to modify their output. The display can provide a presentation screen showing a virtual representation of the output of the device.

A remote-control device can remotely operate the device and can include a display screen offering presentation screens and functionality identical to the display panel. A mobile application can be executed on a user device for remotely operating the device and can generate presentation screens on the user device identical to the display panel.

Light therapy from the device is provided across a plurality of target wavelengths via one or more default sub arrays or user-defined sub arrays.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a perspective view of a light therapy device that is useful for understanding the inventive concepts disclosed herein.

FIG. 2A is an exemplary presentation screen generated by the device for presentation on the display panel, in accordance with one embodiment of the invention.

FIG. 2B is another exemplary presentation screen generated by the device for presentation on the display panel, in accordance with one embodiment of the invention.

FIG. 3 is a simplified block diagram of the system controller of the device, in accordance with one embodiment of the invention.

FIG. 4 is a front view of a remote control of the device, in accordance with one embodiment of the invention.

FIG. 5 is a front view of a smartphone running a mobile application of the device, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

### Definitions

As described herein, a “unit” means a series of identified physical components which are linked together and/or function together to perform a specified function.

As described throughout this document, the term “about” “approximately” “substantially” and “generally” shall be used interchangeably to describe a feature, shape, or measurement of a component within a tolerance such as, for example, manufacturing tolerances, measurement tolerances or the like.

As described herein, a “target wavelength” is used to describe the measurable light/radiation output of one or more optical emitters, and/or the below described array and/or sub array(s).

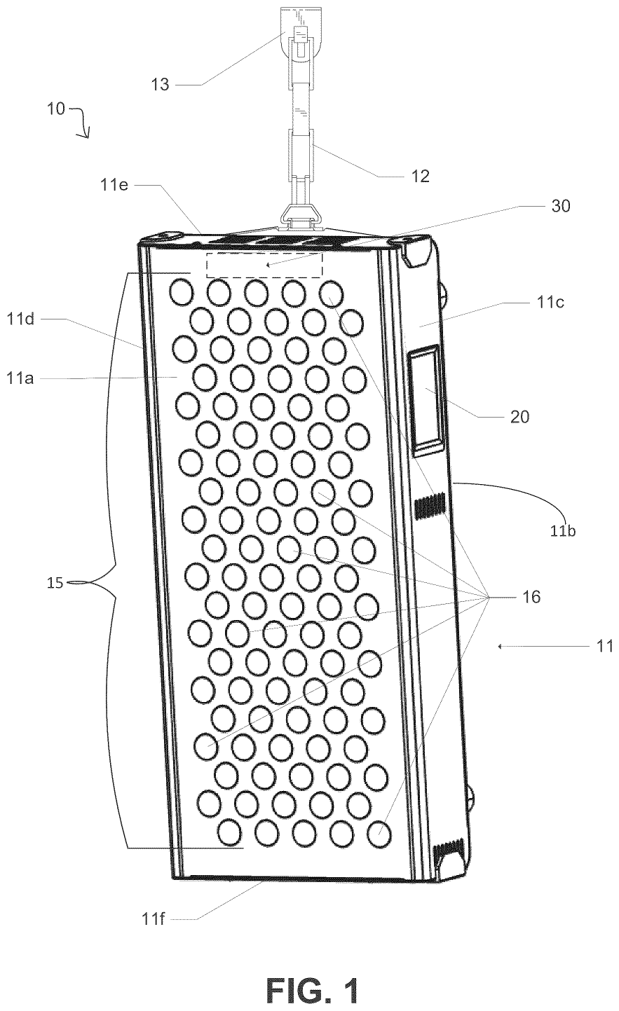

FIGS. 1-5 illustrate one embodiment of a light therapy device 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

As shown and described herein, the light therapy device 10 can include a main body 11 having an array 15 of optical emitters 16 which can operate to produce photonic energy in the form of light/radiation at one or more target wavelengths. The optical emitters can be connected to a display panel 20 and a system controller 30 for allowing a user to selectively control the same.

The main body 11 can function to house the device components and can include a front wall 11a, a back wall 11b, a pair of side walls 11c and 11d, a top wall 11e and a bottom wall 11f that define an interior space. In the preferred embodiment, the main body will be constructed from stamped steel panels and the illustrated rectangular shape having a plurality of air vents 12 along each of the top, bottom, back and side walls.

Of course, other embodiments are contemplated wherein the main body includes any number of different shapes and sizes. Moreover, the main body may be formed from any number of other materials that are, for example, relatively strong and stiff for their weight. Several nonlimiting examples include but are not limited to various metals or metal alloys (e.g., aluminum, titanium, or alloys thereof), plastic/polymers (e.g., high-density polyethylene (HDPE), rigid polyvinyl chloride (PVC), or polyethylene terephthalate (PET)), and/or various composite materials (e.g., carbon fibers in a polymer matrix, fiberglass, etc.).

In one embodiment, a hanging assembly such as the illustrated chain 12 and hook 13 can be provided to allow the device to be suspended from a wall or door, for example. Of course, any number of other connectors such as various clips, brackets, or freestanding stands may also be provided in order to allow a user to position the device in any desirable location and/or orientation for use.

In one embodiment, the array 15 can be positioned along or within the main body and can direct optical emissions produced by each individual emitter 16 outward therefrom in a specified direction. Each of the optical emitters can be communicatively linked to the below described controller 30 in order to receive operating instructions and/or power therefrom.

In one embodiment, each of the optical emitters 16 can comprise a single-color Light Emitting Diode (LED) in the form of a 2-Watt red LED, a 2-Watt blue LED, or a 2-Watt near infrared LED, each operating at 120 volts AC.

In this regard, the emitters comprising a plurality of red LED's can be grouped into a first sub array which produce an output at a first target wavelength of 630 nm which acts to reduce the appearance of fine lines and wrinkles, stimulates hair regrowth and treats. A second sub array of red LED's can produce an output at a second wavelength of about 660 nm which acts to reduce training fatigue by engaging the muscles beneath the skin and reduce neuropathic pain and swelling after injury.

The emitters comprising a plurality of blue LED's can be grouped into a third sub array to produce an output at a third target wavelength of about 480 nm for which acts to repair sun damage to skin, treat chronic skin inflammatory conditions and also treat acne.

The emitters comprising the plurality of near infrared LED's can be grouped into a fourth, fifth sixth and seventh sub array to produce outputs at fourth, fifth, sixth and seventh target wavelengths of about 810 nm, 830 nm, 850 nm and 1060 nm, respectively, which acts to treat brain issues such as traumatic brain injuries, improvement in psychiatric disorders, accelerated would healing, hair growth, bone repair and growth, and enhanced muscle recovery, among other treatments, for example.

In the preferred embodiment, the emitters of the device can be sized and numbered such that the total emissions of the device are represented as follows:
10% of the total optical emissions from the device 10 are produced by the first sub array as visible red light at the first target wavelength of about 630 nm;
39% of the total optical emissions from the device 10 are produced by the second sub array as visible red light at the second target wavelength of about 660 nm;
2% of the total optical emissions from the device 10 are produced by the third sub array as visible blue light at the third target wavelength of about 480 nm;
5% of the total optical emissions from the device 10 are produced by the fourth sub array as near infrared light at the fourth target wavelength of about 810 nm;
5% of the total optical emissions from the device 10 are produced by the fifth sub array as near infrared light at the fifth target wavelength of about 830 nm;
35% of the total optical emissions from the device 10 are produced by the sixth sub array as near infrared light at the sixth target wavelength of about 850 nm, and
4% of the total optical emissions from the device 10 are produced by the seventh sub array as near infrared light at the sixth target wavelength of about 1060 nm.

The above noted ratio and emissions can be provided as the default output of the device and are important for allowing a single device to treat multiple issues as described above during a single session, which is the primary function of the inventive device. In order to accomplish this, the above noted output of the array is specifically chosen as it forms a spectral output that matches the rate of absorption by the human body to trigger physiological responses which achieve the above noted benefits. Stated differently, the overall spectral output of the light produced by the device at the above noted settings specifically matches the coefficient of spectral absorption by the human body, thus allowing the device to achieve results not previously possible.

Although described above with regard to a specific number of emitters operating at specific outputs to treat specific issues, other embodiments are also contemplated. To this end, the device 10 can include any number of individual optical emitters capable of producing light in any color and in any of the visible, near-infrared, infrared, far-infrared and/or other spectrums in order to treat any number of other issues. Moreover, some of the optical emitters within the array can include a different construction than other emitters within the array. Several nonlimiting examples of other types of optical emitters which may be used herein include, but are not limited to heat bulbs, incandescent bulbs, LCD, and/or OLED elements, among others, for example. Such a feature allowing a single device to be capable of performing light therapy at any number of different colors, wavelengths and emission parameters at the same time so as to treat any number of different ailments simultaneously during a single session.

In one embodiment, some or all of the optical emitters 16 within the array 15 can include or comprise multi-color LED's capable of outputting light in a plurality of different colors/wavelengths. Each of these emitters are coupled to the below described controller 30 which can operate the emitters to produce optical outputs that are the same or different from other optical emitters in the array. For example, some of the emitters can produce visible red light at any desirable wavelength suitable for visible red-light therapy, whereas other emitters can produce blue, green, orange or any other color light at any desirable wavelength suitable for any number of different light therapies. In one embodiment, some of the emitters can include, comprise or consist of infrared LED's capable of outputting infrared light at any number of different wavelengths suitable for conducting infrared light therapy in conjunction with the user-defined variable light therapy.

In either instance, the device 10 can include a display panel 20 that is communicatively linked to the system controller 30 in order to allow a user to control the operation of the array 15. In the preferred embodiment, the display panel can include, comprise or consist of a Graphic User Interface (GUI) touch screen device for conducting two-way communication with a user. To this end, the interface can allow the user to instruct the system to perform functions such as transitioning the entire array between an on and off operating state, and/or selectively activating individual sub arrays of emitters (e.g., first sub array, second sub array, third sub array, fourth sub array, fifth sub array, and/or sixth sub array described above), for example.

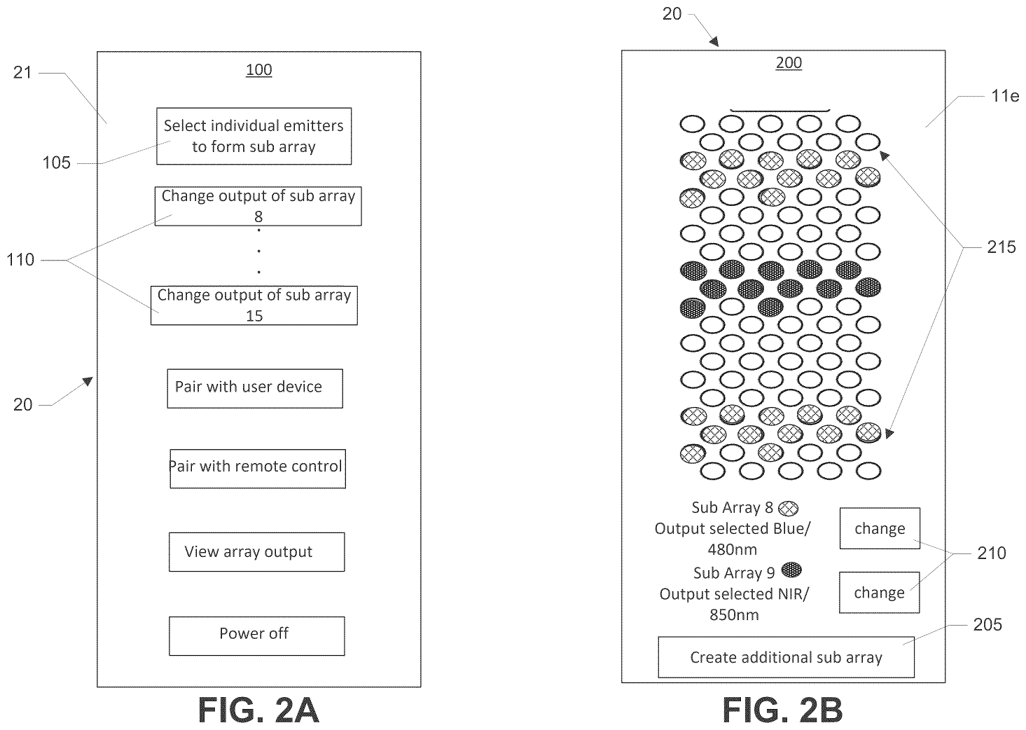

As shown at cutout FIGS. 2A and 2B, the screen 21 of the display panel 20 can generate any number of presentation screens 100 and 200 for allowing a user to individually select 105 any number of individual emitters having multi-color LED'S and to change and control the color/wavelength output of the same 110. Additionally, the display panel can include functionality for allowing a user to group a plurality of individual optical emitters into one or more user-defined sub arrays 205, each having a user-defined color and wavelength output, to change the settings 210 and to see a visual representation of the same on the display panel 215.

As such, the display panel 20 can provide the user with a color matching or other form of visual representation of what the entire array 15 will be outputting when the array is in an operational state. Such a feature is critical, as users are encouraged to never look directly at array of optical emitters during operation. As such, without this feature, the user would have no way to verify the operational state of the array and/or to confirm the actual output is what they intended.

Of course, the display panel is not limited to the use of a touch screen system, as any number of other types of devices such as a combination of non-touch screen displays, buttons, switches and/or other input mechanisms are also contemplated.

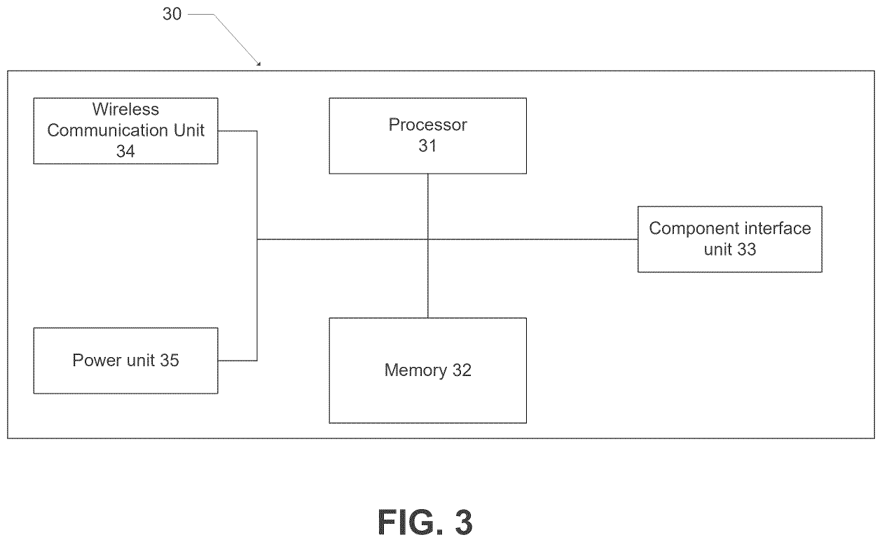

FIG. 3 is a simplistic block diagram illustrating one embodiment of the controller 30 that includes a processing unit 31 that is conventionally connected to an internal memory 32, a component interface unit 33, a wireless communication unit 34, and/or a power unit 35.

Although illustrated as separate elements, those of skill in the art will recognize that one or more system components may comprise or include one or more printed circuit boards (PCB) containing any number of integrated circuit or circuits for completing the activities described herein. The CPU may be one or more integrated circuits having firmware for causing the circuitry to complete the activities described herein. Of course, any number of other analog and/or digital components capable of performing the described functionality can be provided in place of, or in conjunction with the described elements.

The processing unit 31 can include one or more central processing units (CPU) or any other type of device, or multiple devices, capable of manipulating or processing information such as program code stored in the memory 32 in order to allow the device to perform the functionality described herein.

Memory 32 can act to store operating instructions in the form of program code for the processing unit 31 to execute. Although illustrated in FIG. 3 as a single component, memory 32 can include one or more physical memory devices such as, for example, local memory and/or one or more bulk storage devices. As used herein, local memory can refer to random access memory or other non-persistent memory device(s) generally used during actual execution of program code, whereas a bulk storage device can be implemented as a persistent data storage device such as a hard drive, for example. The bulk storage device can contain any number of different programs that permit the processor to perform the functionality described herein and can also receive and store the exercise information for each user.

The component interface unit 33 can function to provide a communicative link between the processing unit 31, the optical emitters 15 and the display panel 20. In this regard, the component interface unit can include any number of different components such as one or more PIC microcontrollers, standard bus, internal bus, connection cables, wireless receiver and/or associated hardware such as USB cables and connectors, and other such hardware capable of linking the various components. Of course, any other means for providing the two-way communication between the system components can also be utilized herein.

The communication unit 34 can include any number of components capable of sending and/or receiving electronic signals with another device, either directly or over a network. In the preferred embodiment, the communication unit 34 can include a Wi-Fi transceiver for communicating directly with a system remote-control device 40 and/or a user device 5 such as a smartphone or tablet running a mobile application 50, for example that can be downloaded onto the user device and installed as an application.

Of course, any number of other transmission and reception mechanisms and protocols can also be utilized herein, several nonlimiting examples include cellular transceivers, Near-Field-Communication (NFC) devices, radio, infrared (IR), RFID, and/or network adapter(s) functioning to communicate over a WAN, LAN, or the internet via an internet service provider, among others, for example.

The power unit 35 can include any number of different components capable of providing the necessary power requirements to each element of the device. In the preferred embodiment, the power unit can include or comprise an A/C electrical power transformer and electrical cord for allowing the device to be plugged into a standard electrical outlet. Of course, other embodiments are contemplated wherein the power unit includes onboard batteries so as to operate without the need to connect to a power outlet.

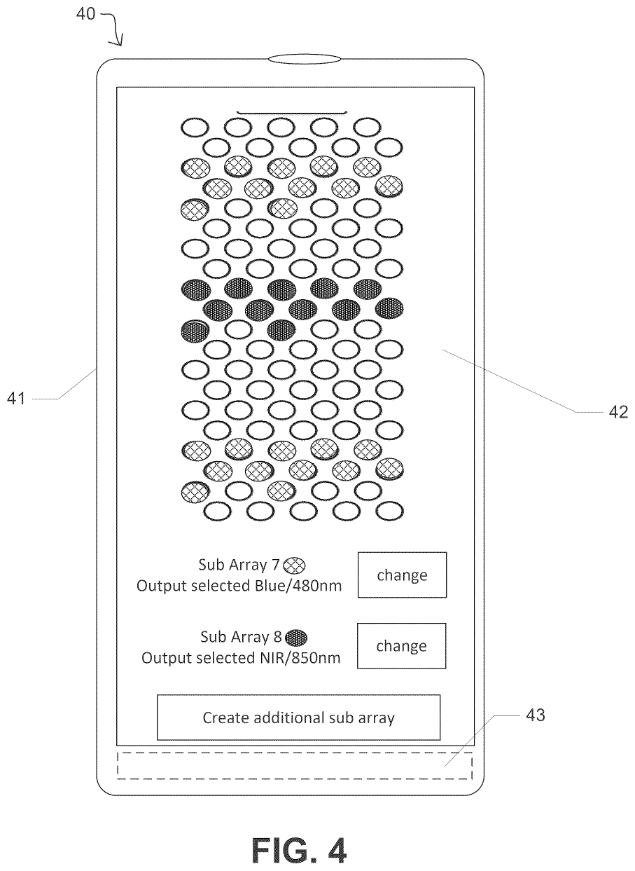

FIG. 4 illustrates one embodiment of a remote-control device 40 that can function to allow a user to selectively control the operation of the device 10 and any additional light therapy devices either individually or as a group. As described herein, the remote-control device can include a main body 41 having a display screen 42 and an onboard controller 43 containing a processor, memory, component interface, and wireless interface, that are substantially identical to that described above with regard to the system controller 30 of the light therapy device.

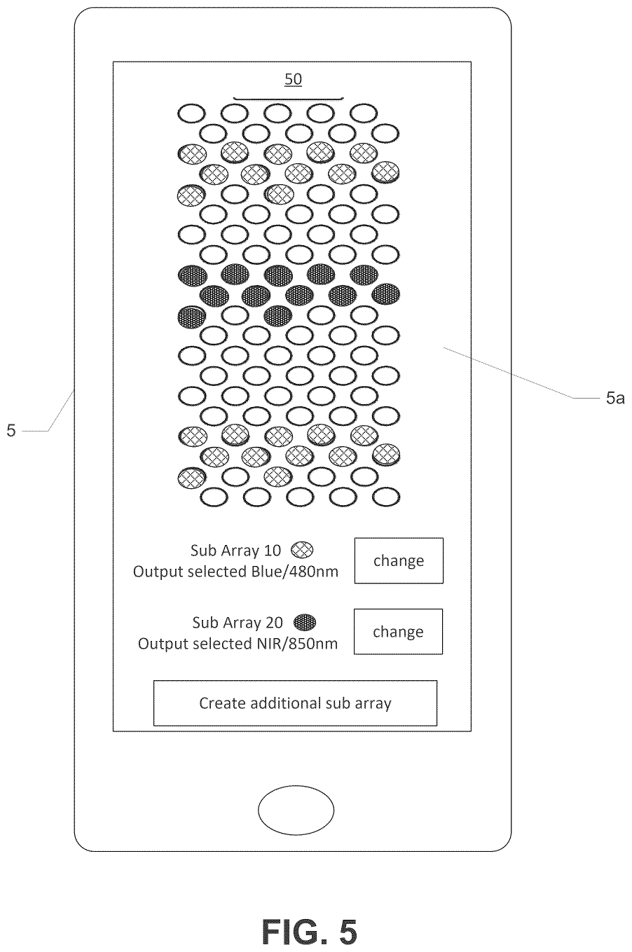

As shown at FIG. 5, the mobile application 50 utilizes the touchscreen 5a, processing capabilities and communication systems of a smartphone 5 or tablet to communicate with the system controller 30 and to control the operation of the device 10 and any additional light therapy devices either individually or as a group.

As described herein, the remote-control device 40 and mobile application 50 can provide a user operating the respective devices with identical functionality as described above with regard to the display panel 20, including the generation of display screens and options for selectively viewing, grouping and controlling each individual emitter of the array to achieve a user defined light therapy session.

In operation, a user can first position a light therapy device in a room via any number of attachment mechanisms such as the above noted hanging assembly and/or a physical stand, for example. Next, the user can activate the display unit 20 to be presented with a plurality of options for configuring and operating the device. For example, the display can provide a settings screen listing the default emissions noted above and a visual representation of the same on the display screen.

If the user is satisfied with the default setting, the user can depress continue on the display screen and the controller will activate the array. Upon activation, the lighting units will provide illumination at the noted levels in order to provide light therapy to the user when the user positions themselves in front of the array.

As noted above, by providing light at a plurality of different colors and spectrums as specifically outlined, the therapy device is able to simultaneously work to reduce the appearance of fine lines and wrinkles, stimulate hair regrowth, reduce training fatigue, reduce neuropathic pain and swelling, repair sun damage to skin, treat chronic skin inflammatory conditions, treat acne, treat brain issues, accelerate would healing, stimulate bone repair and growth, among other treatments, for example.

Because every person's body is different, some users may experience increased health benefits by selectively increasing or decreasing some of the light outputs of the array. As such, if the user wishes to change the settings, they can create any number of different sub arrays.

When this option is selected, the display can provide the user with options for selecting any number of individual lighting elements from the array and providing the desired light output for the selected items. In one embodiment, the display can provide the user with a visual representation of the entire array and the user can select lighting elements by clicking on the corresponding icon on the display representing the lighting element they wish to assign/change/update.

Once the user has created their sub arrays the user can instruct the device to leave any non-selected light elements in the OFF position upon activation of the array, or to allow the controller to automatically activate the non-selected lighting elements in one or more of the colors and intensities described above.

In either instance, once the sub arrays have been created, the display 20 can provide the user with a visual representation of the entire array such that the user can see which lighting elements will be active and at which settings. Such a feature allows the user to confirm the device will operate in the manner they wish, without having to physically look at the actual lighting elements when they are in the ON position.

In one embodiment, the user can utilize a remote control or other device such as a smartphone running the above described mobile application to control the output of the array. When utilized, the controller can send and receive information with the external device so as to permit the display screen of the device to provide the same options as those outlined above. In either instance, the user can save their settings into the memory of the device itself and/or the memory of the remote/smartphone in order to quickly access previous treatments settings for future use.

Additionally, a user can utilize any number of different light therapy devices at one time so as to treat a larger area of their body. Each of the devices can be individually controlled via the devices' onboard controller and display as outlined above or can be individually controlled by a single external device such as the remote control 40 and/or the user device 5 and mobile application 50. In this regard, a user can pair each device 10 to a single external device and can utilize the single remote device to create an unlimited number of sub arrays on each individual light therapy device. Such a feature allows a user to fully customize the type of light therapy they receive along the entire treatment area.

As to a further description of the manner and use of the present invention, the same should be apparent from the above description. Accordingly, no further discussion relating to the manner of usage and operation will be provided.

As described herein, one or more elements of the device 10 can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated

What is claimed is:

1. A method of providing a light therapy treatment, said method comprising:
providing a light therapy device having a main body, an array comprising a plurality of individual optical emitters positioned within the main body, a display panel that is positioned along the main body, and a system controller that is in communication with each of the display panel and the array of optical emitters;
providing, via the display panel, an option to produce an optical output at each of a default output setting and a user-defined output setting,
wherein the default output setting includes a plurality of target wavelengths, and the user-defined output setting includes at least one target wavelength;
receiving, via the display panel an instruction selecting one of the default output setting or the user-defined output setting; and
producing an optical output at the selected output setting.

2. The method of claim 1, wherein the default output setting includes a first target wavelength outputting visible red light at about 630 nm.

3. The method of claim 2, wherein the first target wavelength is outputted by a first sub array containing some of the plurality of optical emitters.

4. The method of claim 1, wherein the default output setting includes a second target wavelength outputting visible red light at about 660 nm.

5. The method of claim 4, wherein the second target wavelength is outputted by a second sub array containing some of the plurality of optical emitters.

6. The method of claim 1, wherein the default output setting includes a third target wavelength outputting visible blue light at about 480 nm.

7. The method of claim 6, wherein the third target wavelength is outputted by a third sub array containing some of the plurality of optical emitters.

8. The method of claim 1, wherein the default output setting includes a fourth target wavelength outputting near infrared light at about 810 nm.

9. The method of claim 8, wherein the fourth target wavelength is outputted by a fourth sub array containing some of the plurality of optical emitters.

10. The method of claim 1, wherein the default output setting includes a fifth target wavelength outputting near infrared light at about 830 nm.

11. The method of claim 10, wherein the fifth target wavelength is outputted by a fifth sub array containing some of the plurality of optical emitters.

12. The method of claim 1, wherein the default output setting includes a sixth target wavelength outputting near infrared light at about 850 nm.

13. The method of claim 12, wherein the sixth target wavelength is outputted by a sixth sub array containing some of the plurality of optical emitters.

14. The method of claim 1, wherein the default output setting includes a seventh target wavelength outputting near infrared light at about 1060 nm.

15. The method of claim 14, wherein the seventh target wavelength is outputted by a seventh sub array containing some of the plurality of optical emitters.

16. The method of claim 1, further comprising:
providing, via the display panel, a visual representation of the selected output setting.

17. The method of claim 1, wherein the display panel comprises a touch screen display, and the user-defined output setting includes
providing, via the display panel, an option to create at least one user defined sub array containing some of the plurality of individual optical emitters.

18. The method of claim 1, wherein each of the plurality of optical emitters of the array comprise single color light emitting diodes.

19. The method of claim 1, wherein each of the plurality of optical emitters of the array comprise one of a single color light emitting diode or a multi-color light emitting diode.
